(19) United States
(12) Patent Application Publication
(10) Pub. No.: US 2006/0223155 A1
(43) Pub. Date: Oct. 5, 2006

(54) Enhancement of in vitro culture or vaccine production in bioreactors using electromagnetic energy

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/339,993

(22) Filed: Jan. 26, 2006

Related U.S. Application Data
(63) Continuation-in-part of application No. 10/700,355, filed on Nov. 3, 2003.
(60) Provisional application No. 60/423,643, filed on Nov. 1, 2002.
(60) Provisional application No. 60/488,490, filed on Jul. 17, 2003.

(51) Int. Cl. C12N 13/00; C12M 1/00

(52) U.S. Cl. 435/173.8; 435/289.1; 435/292.1

(57) ABSTRACT

Disclosed are apparatus and methods for enhancing or improving cell cultures, including cell cultures for the production of monoclonal antibodies, using electromagnetic energy treatment, primarily using electromagnetic radiation in the near infrared to visible region of the spectrum. The delivery of electromagnetic energy to a culture, in accordance with preferred embodiments, enhances or improves the cell culture such as by providing for enhanced and accelerated formation of important biological macromolecules, including, but not limited to, antibodies, proteins, collagen, and polysaccharides, and also providing for accelerated cellular replication and an enhancement or prolongation of the life of cells so treated.

### CLAIM OF PRIORITY

This application is a continuation-in-part from U.S. patent application Ser. No. 10/700,355, filed Nov. 3, 2003, which is incorporated in its entirety by reference herein, and which claims priority under 35 U.S.C. § 119(e) to U.S. Provisional Patent Application Nos. 60/423,643 filed Nov. 1, 2002 and 60/488,490 filed Jul. 17, 2003, the disclosures of which are hereby incorporated by reference in their entireties.

### BACKGROUND OF THE INVENTION

1. Field of the Invention

The present invention relates in general to enhancing cell cultures, and more particularly, to novel apparatus and methods for enhancing production of cells or cell-derived products in bioreactors through application of electromagnetic energy.

2. Description of the Related Art

In vitro cell cultures are used in a variety of contexts, including biotechnology. Many methods for culturing cells involve bioreactors, of which there are myriad well-known varieties. In general, bioreactors provide an environment conducive to cell growth and productivity by controlling such variables as the pH, oxygen, or carbon dioxide levels experienced by the cells. Bioreactors provide nutrients to the cell cultures, and generally agitate the cultures for purposes of aeration using such methods as rocking, stirring, or channeling fluid or gas through the culture. Bioreactors are used for diverse purposes and on diverse scales. For example, small-scale bioreactors may be used on desktops in research laboratories, while large-scale bioreactors may be used in industrial pharmaceutical plants. Important uses of bioreactors include the culturing of bacteria or hybridomas for the large-scale production of macromolecules such as antibodies or other proteins that are useful as biotechnological drugs, the culturing of bacteria useful for vaccines, and culturing of animal cells containing viruses useful for biotechnology or vaccines. Obtaining a drug agent or vaccine material via bioreactors can be expensive, especially as compared to many synthetic methods used for small molecule pharmaceuticals. As a result, there is a need for a method to increase.the yield and efficacy of bioreactors.

### SUMMARY OF THE INVENTION

In certain embodiments, a bioreactor comprises a reservoir for holding a cell culture comprising cells and a culture medium. The bioreactor further comprises an electromagnetic radiation source which irradiates the cells with electromagnetic radiation having a power density above about 1 mW/cm² within a wavelength bandwidth of less than or equal to approximately 100 nanometers.

In certain embodiments, a method enhances the production of cells or cell-derived products from a bioreactor containing a cell culture. The method comprises delivering an effective amount of electromagnetic energy to cells in the cell culture. Delivering the effective amount of electromagnetic energy includes delivering electromagnetic radiation having a power density of at least about 1 mW/cm² within a wavelength bandwidth of less than or equal to approximately 100 nanometers to the cells in the cell culture.

In certain embodiments, a method enhances the production of a vaccine from a bioreactor containing cells in a cell culture. The method comprises delivering an effective amount of electromagnetic energy to cells in the cell culture. Delivering the effective amount of electromagnetic energy includes delivering electromagnetic radiation having a power density of at least about 1 mW/cm² within a wavelength bandwidth of less than or equal to approximately 100 nanometers.

For purposes of summarizing the present invention, certain aspects, advantages, and novel features of the present invention have been described herein above. It is to be understood, however, that not necessarily all such advantages may be achieved in accordance with any particular embodiment of the present invention. Thus, the present invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 schematically illustrates an exemplary bioreactor equipped with an electromagnetic radiation source for illuminating a cell culture.

FIG. 2 schematically illustrates an exemplary rocking bag bioreactor system equipped with an electromagnetic radiation source for illuminating a cell culture.

FIG. 3 schematically illustrates another exemplary bioreactor comprising a conduit for cycling a cell culture, wherein the conduit is equipped with an electromagnetic radiation source for illuminating the cell culture.

FIGS. 4A and 4B schematically illustrate two embodiments of a blanket which emits electromagnetic radiation for illuminating a cell culture.

FIG. 5 schematically illustrates a bioreactor equipped with a blanket which emits electromagnetic radiation for illuminating a cell culture.

FIG. 6 schematically illustrates a rocking bag bioreactor equipped with a blanket which emits electromagnetic radiation for illuminating a cell culture.

FIG. 7 is a block diagram of a control circuit comprising a programmable controller.

### DETAILED DESCRIPTION OF THE PREFERRED EMBODIMENTS

Methods for enhancing the performance of cell cultures using electromagnetic energy are based in part on the discovery that electromagnetic energy applied to a culture enhances or improves the cell culture. In certain embodiments, irradiation of the cells within the cell culture facilitates enhanced and accelerated formation of important biological macromolecules, including, but not limited to, antibodies, proteins, collagen, and polysaccharides. In certain embodiments, irradiation of the cells also facilitates accelerated cellular replication or an enhancement or prolongation of the life of cells so irradiated. Methods disclosed in accordance with certain embodiments described herein may be used to accelerate the production of vaccines and/or other important products containing biological materials.

The term “cell” as used herein is a broad term used in its ordinary sense and includes animal cells such as human or mammalian cells, hybridomas, and single-celled organisms such as bacteria. A “cell culture” includes one or more cells in a medium that provides for the growth of the one or more cells. The term “bioreactor” as used herein is a broad term used in its ordinary sense, and may be of any type, including those designed for small-scale cultures such as are performed in small containers as are commonly used in research laboratories, as well as large-scale bioreactors comprising vessels or vats as are commonly used in the pharmaceutical and biotech industries to produce and harvest biological macromolecules on a pilot plant or commercial scale.

Terms such as “enhancement” or “enhance” as used with regard to the performance of cells or cell cultures refer to an improvement of properties of the culture or cells as compared to a culture or cells that are not irradiated, such improved properties including enhanced and accelerated formation of important biological macromolecules, including, but not limited to, antibodies, proteins, vaccines, collagen, and polysaccharides by the cell, accelerated cellular replication, and prolongation of the life of the cell or cells.

In certain embodiments, an electromagnetic radiation source is provided for enhancing the performance of a cell culture in a bioreactor by providing an effective amount of electromagnetic energy to the cell culture. Various forms of electromagnetic energy are compatible with certain embodiments described herein, including but not limited to, visible light, infrared (IR) light (e.g., mid-IR, long-IR), radiofrequency (RF) radiation, electric fields, and magnetic fields.

In certain embodiments, the precise power density of the electromagnetic energy selected depends on a number of factors, including the specific wavelength or range of wavelengths selected, the type of cells, the particular macromolecule(s) or cell behavior desired, the medium, and the like. For example, when the cell culture is in a bioreactor having a large volume, one may take into account attenuation of the energy of the electromagnetic radiation as it travels through the culture medium to reach cells at a greater distance from the source. If, however, the culture is stirred or similarly manipulated, the need to account for attenuation may be obviated in that all cells in the culture will receive substantially equal energy. Similarly, it should be understood that the power density of electromagnetic energy to be delivered to the culture may be adjusted to be combined with any other culture-enhancing or therapeutic agents to achieve a desired biological effect. The selected power density will again depend on a number of factors, including the specific electromagnetic energy wavelength chosen, the individual additional agent or agents chosen, and the cell line used.

In certain embodiments, the source may be a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together with references contained therein.

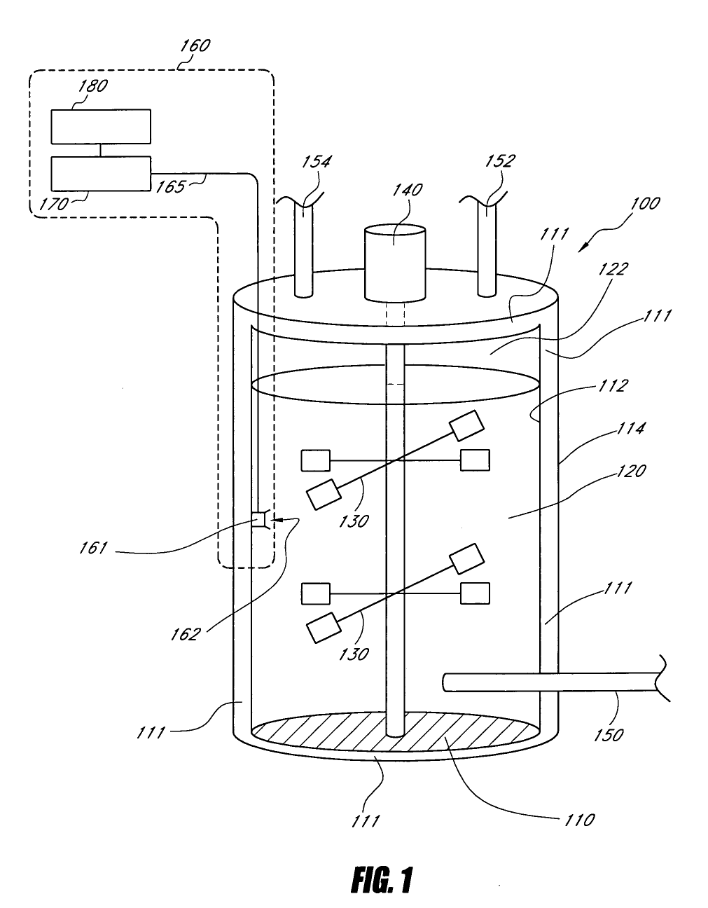

FIG. 1 schematically illustrates an exemplary bioreactor 100, comprising a reservoir 110 and a cell culture 120 contained in the reservoir 110. The reservoir 110 has one or more walls 111, each wall 111 having an interior surface 112 and an exterior surface 114. In certain embodiments, the walls 111 are composed of an opaque material, such as metal. In other embodiments, at least a portion of the walls 111 of the reservoir 110 is composed of a transparent or translucent material, such as plastic or glass. The reservoir 110 can be substantially cylindrical, as shown, or can assume any other shape for holding a cell culture 120. The cell culture 120 comprises cells and a culture medium.

The bioreactor 100 schematically illustrated by FIG. 1 further comprises one or more impellers 130 and a motor 140 coupled to the impellers 130 for agitating the cell culture 120. The bioreactor 100 schematically illustrated by FIG. 1 further comprises a gas inlet 150 for adding a gas or gases to the cell culture 120, a gas outlet 152 for removing a gas or gases from the cell culture 120, and one or more liquid conduits 154 for adding to and/or removing from the cell culture 120 a liquid, liquids, nutrients, or other materials. The bioreactor 100 further comprises at least one electromagnetic radiation source 160 for irradiating the cell culture 120. The source 160 has an emitter 161 with an output emission area 162 positioned to irradiate a portion of the cell culture 120 with an effective power density and wavelength of electromagnetic radiation. The source 160 of certain embodiments further comprises at least one power conduit 165 coupled to the emitter 161, a power source 170 coupled to the power conduit 165, and a control circuit 180 coupled to the power source 170.

In certain embodiments, the emitter 161 is within the reservoir 110 (e.g., fixedly or movably attached to the interior surface 112 of a wall 111 of the reservoir 110 or another structure within the reservoir 110), as, for example, where the walls 111 are opaque. In other embodiments, the emitter 161 is outside the reservoir 110 (e.g., fixedly or movably attached to the exterior surface 114 of a wall 111 of the reservoir 110 or another structure outside the reservoir 110), as, for example, where the wall 111 is transparent or translucent. In certain embodiments in which the reservoir 110 has a wall 111 which is either transparent or translucent, the emitter 161 may be positioned a distance from the exterior surface 114. In other embodiments, the emitter 161 is fixedly attached between the interior surface 112 and the exterior surface 114 of a wall 111 of the reservoir 110. Additional embodiments provide a plurality of emitters 161 that are inside the reservoir 110, outside the reservoir 110, or part of the walls 111 of the reservoir 110. Other embodiments provide a plurality of emitters 161 that are fixedly or movably attached to any combination of the interior surface 112, the exterior surface 114, the space between the interior surface 112 and exterior surface 114, and other structures (e.g., plates or panels) which are spaced from the walls 111 of the reservoir 110.

In certain embodiments, the emitter 161 is situated to irradiate the cell culture 120 from a position within the culture 120. For example, as schematically illustrated in FIG. 1, the emitter 161 may be immersed within the cell culture 120. In this manner, electromagnetic radiation emitted from the emitter 121 does not propagate through another medium, such as air, prior to irradiating the cell culture 120. In other embodiments, the emitter 161 is situated such that the electromagnetic radiation emitted from the emitter 161 does propagate through another medium prior to irradiating the cell culture 120. For example, the emitter 161 can be positioned to be within the reservoir 110 but outside the cell culture 120 (e.g., above the cell culture 120 in region 122).

In certain embodiments, the power conduit 165 comprises an electrical conduit which transmits electrical signals and power to the emitter 161 (e.g., laser diode or light-emitting diode). In certain embodiments, the power conduit 165 comprises an optical conduit (e.g., optical waveguide) which transmits optical signals and power to the emitter 161 (e.g., output end of the optical conduit) which emits electromagnetic radiation into an output emission area 162. In certain such embodiments, the emitter 161 comprises various optical elements (e.g., lenses, diffusers, and/or waveguides) which transmit at least a portion of the optical power received via the power conduit 165. As schematically illustrated in FIG. 1, the power conduit 165, the power source 170, and the control circuit 180 are outside the reservoir 110. In still other embodiments, at least one of the power conduit 165, the power source 170, and the control circuit 180 is within the reservoir 110. While FIG. 1 schematically illustrates the emitter 161, the power conduit 165, the power source 170, and the control circuit 180 as being separate from one another, in certain embodiments, two or more of these components are integral with one another. For example, in certain embodiments, the control circuit 180 and the power source 170 are components of a single electromagnetic radiation source controller.

It is conceived that any combination of the above-described configurations of the emitter 161 or plurality of emitters 161 is compatible with various embodiments described herein. Furthermore, FIG. 1 is merely illustrative of an exemplary bioreactor configuration compatible with certain embodiments described herein. Other certain embodiments utilize emitters 161 coupled to bioreactors comprising other elements or to bioreactors of entirely different configurations.

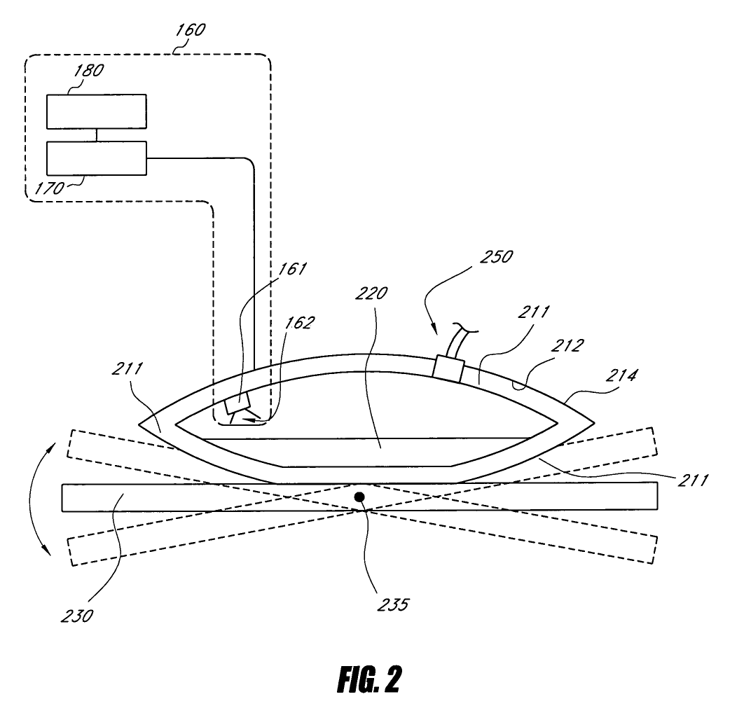

FIG. 2 schematically illustrates another exemplary bioreactor 200. The bioreactor 200 comprises a reservoir 210 and a cell culture 220 within the reservoir 210. The reservoir 210 has one or more walls 211, each wall 211 having an interior surface 212 and an exterior surface 214. The cell culture 220 comprises cells and a culture medium. The walls 211 of the reservoir 210 comprise flexible plastic and the reservoir 210 rests on a platform 230 that rocks the reservoir 210 by cyclically rotating through small angles about an axis 235. Such a reservoir 210 is commonly known in the art as a rocking bag system. The rocking motion agitates the cell culture 220. In certain embodiments, the bioreactor 200 comprises an apparatus 250 for regulating the cell culture environment. The apparatus 250 may comprise a series of input and output valves for adding or removing nutrients, gases, liquids, and so forth, and sensors of various parameters of the cell culture environment (e.g., pH, temperature). In certain embodiments, the bioreactor 200 further comprises one or more emitters 161 positioned on or within the interior surface 212, on or some distance away from the exterior surface 214, or between the interior surface 212 and the exterior surface 214 in configurations similar to those described with respect to FIG. 1.

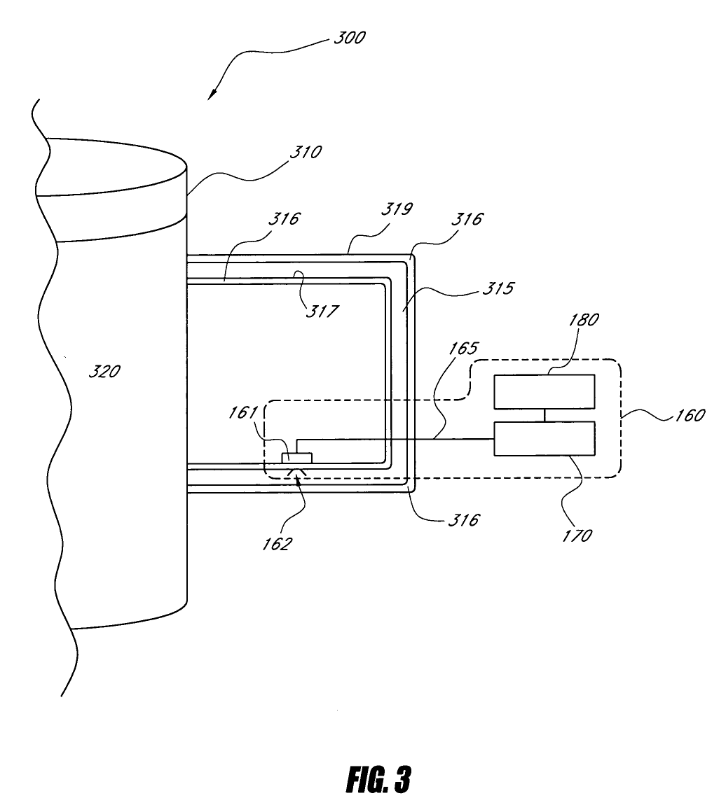

FIG. 3 schematically illustrates another exemplary bioreactor 300 comprising a reservoir 310 and a conduit 315. The conduit 315 has one or more walls 316, each wall 316 having an interior surface 317 and an exterior surface 319. The bioreactor 300 further comprises one or more emitters 161 having an output emission area 162 positioned to irradiate a portion of the cell culture 320 located within the conduit 315 with an effective power density and wavelength of electromagnetic radiation. The emitter 161 or a plurality thereof may be positioned on or within the interior surface 317 of the conduit 315, on or some distance away from the exterior surface 319 of the conduit 315, or between the interior surface 317 and the exterior surface 319 of the conduit 315. A cell culture 320 within the reservoir 310 is cycled through the conduit 315 such that at least a portion of the cell culture 320 is removed from the reservoir 310, irradiated by the source 160, and returned to the reservoir 310. In certain embodiments in which the cycle rate affects the power density applied to the cells, the cycle rate is optimized.

The source 160 preferably generates and emits electromagnetic radiation in the visible to near-infrared wavelength range. In certain embodiments, the emitter 161 comprises one or more laser diodes, which each provide coherent electromagnetic radiation. In embodiments in which the electromagnetic radiation from the emitter 161 is coherent, the emitted electromagnetic radiation may produce “speckling” due to coherent interference of the electromagnetic radiation. This speckling comprises intensity spikes which are created by constructive interference. For example, while the average power density may be approximately 10 mW/cm², the power density of one such intensity spike in proximity to the cells being irradiated may be approximately 300 mW/cm². In certain embodiments, this increased power density due to speckling can improve the efficacy of applications of coherent electromagnetic radiation over those of incoherent electromagnetic radiation for illumination deeper into the cell culture of large bioreactors.

In other embodiments, the emitter 161 provides incoherent electromagnetic radiation. Exemplary emitters 161 of incoherent electromagnetic radiation include, but are not limited to, incandescent lamps or light-emitting diodes. A heat sink can be used with the emitter 161 (for either coherent or incoherent sources) to remove heat from the source 160 and to inhibit temperature increases in the cell culture 120 in the bioreactor. Some embodiments use a combination of coherent and incoherent electromagnetic radiation emitters 161.

In certain embodiments, the source 160 generates electromagnetic radiation which is substantially monochromatic (i.e., electromagnetic radiation having one wavelength, or electromagnetic radiation having a narrow band of wavelengths). In certain embodiments, the source 160 generates electromagnetic energy having a power density above about 1 mW/cm² within a wavelength bandwidth of approximately 100 nanometers or less. For example, in certain embodiments in which the source 160 comprises a laser, the wavelength bandwidth is less than or equal to approximately 10 nanometers, and in certain other embodiments in which the source 160 comprises a light-emitting diode, the wavelength bandwidth is less than or equal to approximately 80 nanometers. In certain embodiments, the electromagnetic radiation has one or more wavelengths between approximately 400 nanometers and approximately 4 microns.

To maximize the amount of electromagnetic radiation transmitted to the cell culture 120, the wavelength of the electromagnetic radiation is selected in certain embodiments to be at or near a transmission peak (or at or near an absorption minimum) of the cell culture 120. In certain such embodiments, the wavelength corresponds to a peak in the transmission spectrum at about 820 nanometers. In certain embodiments, the wavelength of the electromagnetic radiation is between about 630 nanometers and about 1064 nanometers, while in certain other embodiments, the electromagnetic radiation has one or more wavelengths between about 630 nanometers and about 910 nanometers. The electromagnetic radiation in still other embodiments has one or more wavelengths between about 780 nanometers and about 840 nanometers (e.g., wavelengths of about 790, 800, 810, 820, or 830 nanometers). In certain embodiments, the electromagnetic radiation has one or more wavelengths between about 800 nanometers and about 815 nanometers. In still other embodiments in which the cell culture contains water, the electromagnetic radiation has one or more wavelengths between approximately 1.3 microns and approximately 2.9 microns.

In other embodiments, the source 160 generates electromagnetic radiation having a plurality of wavelengths. In certain such embodiments, each wavelength is selected so as to work with one or more chromophores within the cells of the culture. Without being bound by theory or a particular mechanism, in certain embodiments, irradiation of chromophores increases the production of ATP in the cells, thereby producing beneficial effects. In certain embodiments, the source 160 is adapted to generate electromagnetic radiation in a first wavelength range and electromagnetic radiation in a second wavelength range. For example, in certain embodiments, electromagnetic radiation in a visible or infrared wavelength range is applied concurrently with electromagnetic radiation in a radio-frequency (RF) range. In certain other embodiments, the source 160 is adapted to generate electromagnetic radiation in a first wavelength range sequentially with electromagnetic radiation in a second wavelength range. In certain embodiments, the source 160 is adapted to generate electromagnetic radiation and a magnetic field, both of which are applied to the cell culture, either concurrently or sequentially.

In certain embodiments, the source 160 includes at least one continuously emitting GaAlAs laser diode having a wavelength of about 830 nanometers. In another embodiment, the source 160 comprises a laser source having a wavelength of about 808 nanometers. In still other embodiments, the source 160 includes at least one vertical cavity surface-emitting laser (VCSEL) diode. Other sources 160 compatible with embodiments described herein include, but are not limited to, light-emitting diodes (LEDs) and filtered lamps.

The source 160 is capable of emitting electromagnetic energy at a power sufficient to achieve a predetermined power density in the output emission area 162 within the cell culture. Without being bound by theory or a particular mechanism, in certain embodiments, application of electromagnetic radiation to cell cultures is advantageously effective when irradiating the cell culture with power densities of electromagnetic radiation within a selected wavelength range (e.g., between about 630 nanometers and about 910 nanometers) of at least about 1 mW/cm² and up to about 1 W/cm². In various embodiments, the power density within the selected wavelength range is at least about 1, 5, 10, 15, 20, 30, 40, 50, 60, 70, 80, or 90 mW/cm², respectively, depending on the desired performance of the cell culture. In various embodiments, the power density within the selected wavelength range is about 1 mW/cm² to about 100 mW/cm², about 1 mW/cm² to about 15 mW/cm², or about 2 mW/cm² to about 20 mW/cm², respectively, depending on the desired performance of the cell culture. Without being bound by theory or a particular mechanism, in certain embodiments, these power densities are especially effective at producing the desired biostimulative effects on the cultures being irradiated. To achieve efficacious power densities, the source 160 emits electromagnetic energy having a total power output of about 0.1 mW to about 500 mW, including about 0.5, 1, 5, 10, 20, 30, 50, 75, 100, 150, 200, 250, 300, and 400 mW, but may also be up to about 1000 mW.

In certain embodiments, the power density of electromagnetic radiation within the selected wavelength range is substantially above the power density available using sunlight as the electromagnetic radiation. For example, the irradiance of sunlight between approximately 750 nanometers and approximately 850 nanometers is approximately 0.01 mW/cm², which is-quite low and unlikely to create any beneficial effect. Collecting sunlight over a larger area and focusing the collected sunlight to a smaller area can increase the power density in the selected wavelength range beyond the available non-focused levels. However, such focusing would also produce higher power densities outside the selected wavelength range (e.g., above 1 micron), thereby generating significant unwanted heating.

Taking into account the attenuation of energy as it propagates through a cell culture, in certain embodiments, power densities at the surface of the cell culture on which the electromagnetic radiation impinges (hereafter referred to as the “surface of the cell culture”) are selected to be sufficiently high so as to attain the selected power densities for cells on the interior of the culture. To achieve such power densities at the surface of the cell culture, the source 160 is preferably capable of emitting electromagnetic energy having a total power output of at least about 25 mW to about 100 W. An upper limit of the power density at the surface is defined to be the power density at which cell damage occurs. In various embodiments, the total power output is limited to be no more than about 30, 50, 75, 100, 150, 200, 250, 300, 400, or 500 mW, respectively. In certain embodiments, the source 160 comprises a plurality of sources used in combination to provide the total power output. The actual power output of the source 160 is preferably controllably variable. In this way, the power of the electromagnetic energy emitted can be adjusted in accordance with a selected power density irradiating target cells within the culture.

Certain embodiments utilize a source 160 that includes only a single laser diode that is capable of providing about 25 mW to about 100 W of total power output. In certain such embodiments, the laser diode can be optically coupled to the cell culture via an optical fiber or can be configured to provide a sufficiently large spot size to avoid power densities which would bum or otherwise damage the cells of the cell culture. In other embodiments, the source 160 utilizes a plurality of sources (e.g., laser diodes) arranged in a grid or array that together are capable of providing at least about 25 mW to about 100 W of total power output. The source 160 of other embodiments may also comprise sources having power capacities, wavelengths, or other properties outside of the limits set forth above.

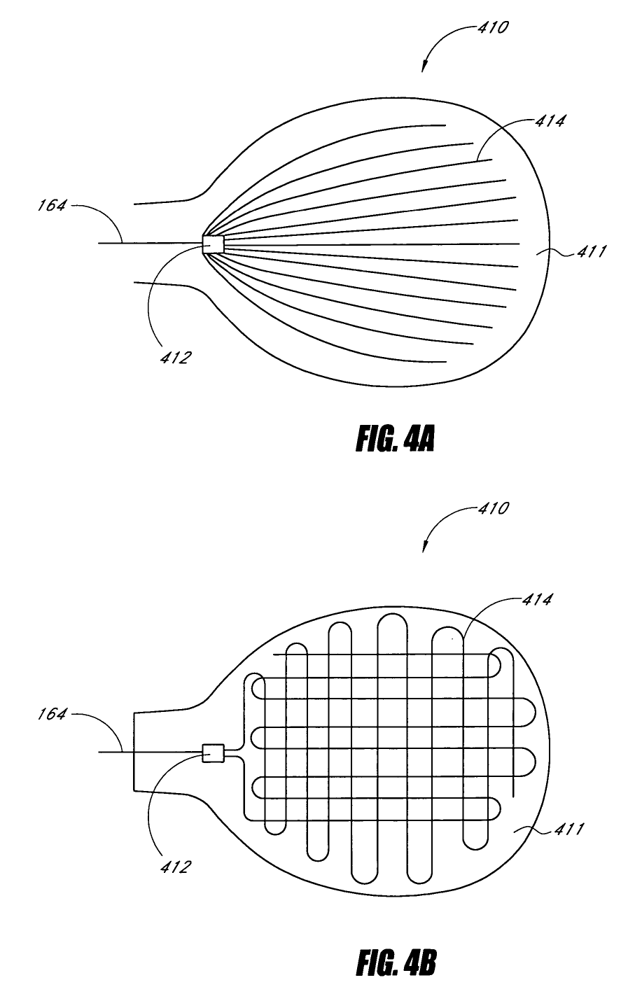

FIGS. 4A and 4B schematically illustrate an exemplary source 160 comprising a blanket 410 which emits electromagnetic radiation. FIG. 4A schematically illustrates an embodiment of the blanket 410 comprising a flexible substrate 411 (e.g., flexible circuit board), a power conduit interface 412, and a sheet formed by optical fibers 414 positioned in a fan-like configuration. FIG. 4B schematically illustrates an embodiment of the blanket 410 comprising a flexible substrate 411, a power conduit interface 412, and a sheet formed by optical fibers 414 woven into a mesh. In certain embodiments, the blanket 410 is positioned within the reservoir of a bioreactor so as to cover an area of a cell culture to which electromagnetic radiation is to be applied.

In certain such embodiments, the power conduit interface 412 is coupled to an optical fiber conduit 164 which provides optical power to the blanket 410. The optical power interface 412 of certain embodiments comprises a beam splitter or other optical device which distributes the incoming optical power among the various optical fibers 414. In other embodiments, the power conduit interface 412 is coupled to an electrical conduit which provides electrical power to the blanket 410. In certain such embodiments, the power conduit interface 412 comprises one or more laser diodes, the output of which is distributed among the various optical fibers 414 of the blanket 410. In certain other embodiments, the blanket 410 comprises an electroluminescent sheet which responds to electrical signals from the power conduit interface 412 by emitting electromagnetic radiation. In such embodiments, the power conduit interface 412 comprises circuitry which distributes the electrical signals to appropriate portions of the electroluminescent sheet.

The side of the blanket 410 nearer a cell culture, in certain embodiments, has an electromagnetic radiation scattering surface, such as a roughened surface to increase the amount of electromagnetic radiation scattered out of the blanket 410 towards the culture. In certain embodiments, the side of the blanket 410 further from the culture is covered by a reflective coating so that electromagnetic radiation emitted away from the culture is reflected back towards the culture. This configuration is similar to configurations used for the “back illumination” of liquid-crystal displays (LCDs). Other configurations of the blanket 410 are compatible with embodiments described herein.

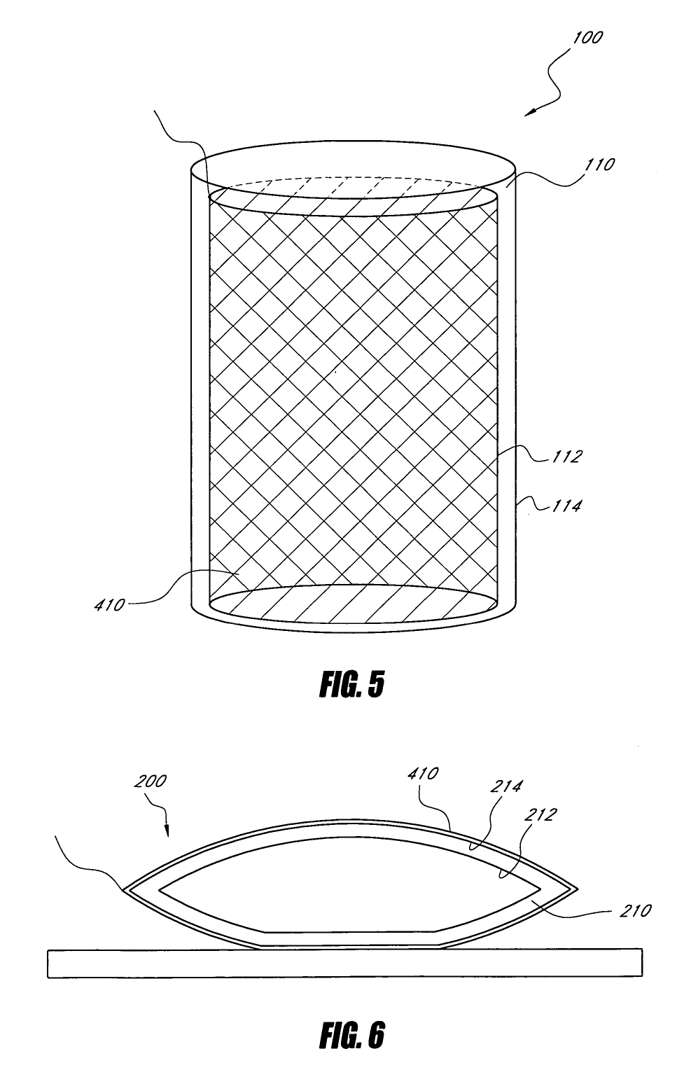

FIG. 5 schematically illustrates an exemplary bioreactor 100 equipped with a source 160 comprising a blanket 410 which emits electromagnetic radiation. The blanket 410 covers at least a portion of the interior surface 112 of the reservoir 110. In certain embodiments, the blanket 410 covers a substantial portion of the interior surface 112 of the reservoir 110, as schematically illustrated by FIG. 5. In other embodiments, the blanket 410 covers at least a transparent or translucent portion of the exterior surface 114 of the reservoir 110. In other embodiments, the blanket 410 is integrated with the reservoir 110 such that it is located between the interior surface 112 and the exterior surface 114 thereof.

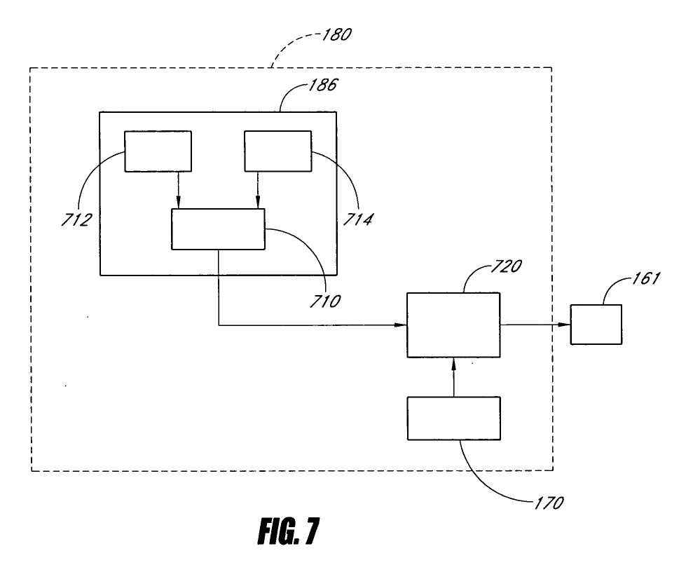

FIG. 6 schematically illustrates another bioreactor 200 equipped with a source 160 comprising a blanket 410. The bioreactor 200 of FIG. 6 is a rocking bag system. In certain embodiments, the blanket 410 covers at least a portion of the interior surface 212 of the reservoir 210. In other embodiments, the blanket 410 covers at least a transparent or translucent portion of the exterior surface 214 of the reservoir 210. In still other embodiments, the blanket 410 is integrated with the reservoir 210 such that at least a portion thereof is disposed between the interior surface 212 and the exterior surface 214 thereof.

FIG. 7 is a block diagram of a control circuit 180 operatively coupled to the emitter 161 and comprising the power source 170 and a programmable controller 186 according to certain embodiments described herein. The control circuit 180 is configured to adjust the power of the electromagnetic energy emitted by the emitter 161 to generate a selected power density at the cell culture.

In certain embodiments, the programmable controller 186 comprises a logic circuit 710, a clock 712 coupled to the logic circuit 710, and an interface 714 coupled to the logic circuit 710. The clock 712 of certain embodiments provides a timing signal to the logic circuit 710 so that the logic circuit 710 can monitor and control timing intervals of the applied electromagnetic radiation. Examples of timing intervals include, but are not limited to, total irradiation times, pulsewidth times for pulses of applied electromagnetic radiation, and time intervals between pulses of applied electromagnetic radiation. In certain embodiments, one or more emitters 161 can be selectively turned on and off to reduce the thermal load on the cells and to deliver a selected power density to particular areas of the culture.

The interface 714 of certain embodiments provides signals to the logic circuit 710 which the logic circuit 710 uses to control the applied electromagnetic radiation. The interface 714 can comprise a user interface or an interface to a sensor monitoring at least one parameter of the electromagnetic radiation application. In certain such embodiments, the programmable controller 186 is responsive to signals from the sensor to preferably adjust the electromagnetic radiation application parameters to optimize the measured response. The programmable controller 186 can thus provide closed-loop monitoring and adjustment of various irradiation parameters to optimize the photo-assisted processes. The signals provided by the interface 714 from a user are indicative of parameters that may include, but are not limited to, cell culture characteristics (e.g., reflectivity, color, etc.), selected applied power densities, target time intervals, and power density/timing profiles for the applied electromagnetic radiation.

In certain embodiments, the logic circuit 710 is coupled to a source driver 720. The source driver 720 is coupled to the power source 170, which in certain embodiments comprises a battery and in other embodiments comprises an alternating current source. The source driver 720 is also coupled to the emitter 161. The logic circuit 710 is responsive to the signal from the clock 712 and to user input from the user interface 714 to transmit a control signal to the source driver 720. In response to the control signal from the logic circuit 710, the source driver 720 adjusts and controls the power applied to the emitter 161. Other control circuits besides the control circuit 700 of FIG. 7 are compatible with embodiments described herein.

In certain embodiments, the logic circuit 710 is responsive to signals from a sensor monitoring at least one parameter of the electromagnetic radiation application to control the applied electromagnetic radiation. For example, certain embodiments comprise a temperature sensor thermally coupled to the cell culture to provide information regarding the temperature of the culture to the logic circuit 710. In such embodiments, the logic circuit 710 is responsive to the information from the temperature sensor to transmit a control signal to the source driver 720 so as to adjust the parameters of the applied electromagnetic radiation to maintain the temperature below a predetermined level.

During the application of electromagnetic energy to the cell culture, the electromagnetic energy may be pulsed or it may be continuously provided. If the electromagnetic radiation is pulsed, the pulses for treatment may be at least about 1 microsecond long and occur at a frequency of up to about 100 kHz. Time between pulses may be longer or shorter than the time of the pulse, and can vary, for example, from a few nanoseconds to several seconds or minutes.

In certain embodiments, the application of electromagnetic energy proceeds continuously for anywhere from a few seconds to several hours, days or weeks. In some embodiments, the application lasts for a period of about 10 seconds to about 2 hours. In other embodiments, the application lasts for a period of about 30 seconds to about 2 hours. In still other embodiments, the application proceeds continuously for a period of about 1 minute to about 10 minutes. In some embodiments, the application proceeds for a period of about 1 minute to about 5 minutes. In other embodiments, the electromagnetic energy is delivered for at least one application period of at least about five minutes. In still other embodiments at least one application period of at least about ten minutes is used.

In certain embodiments, the application may be terminated after one application period, while in other embodiments, the application may be repeated for at least two application periods. If there is more than one application period, the time between application periods can be from one or more hours to several days. In certain embodiments, the time between subsequent application periods is at least about five minutes; in other embodiments, the time between subsequent application periods is at least about 1 to 2 days; in still other embodiments, the time between subsequent application periods is at least about one week. In one embodiment, the application is divided into at least ten periods, each period lasting about one hour during which the electromagnetic radiation is delivered in a series of pulses, with a time of at least about six hours passing between the application periods.

The explanations and illustrations presented herein are intended to acquaint others skilled in the art with various embodiments of the invention, its principles, and its practical application. Those skilled in the art may adapt and apply the invention in its numerous forms, as may be best suited to the requirements of a particular use. Accordingly, the specific embodiments of the present invention as set forth herein are not intended as being exhaustive or limiting of the invention.

What is claimed is:

1. A bioreactor, comprising:
a reservoir for holding a cell culture comprising cells and a culture medium; and
an electromagnetic radiation source which irradiates the cells with electromagnetic radiation having a power density above about 1 mW/cm² within a wavelength bandwidth of less than or equal to approximately 100 nanometers.

2. The bioreactor of claim 1, wherein the wavelength bandwidth is less than or equal to approximately 80 nanometers.

3. The bioreactor of claim 1, wherein the wavelength bandwidth is less than or equal to approximately 10 nanometers.

4. The bioreactor of claim 1, wherein the electromagnetic radiation has one or more wavelengths between about 400 nanometers and about 4 microns.

5. The bioreactor of claim 1, wherein the electromagnetic radiation has one or more wavelengths between about 630 nanometers and about 910 nanometers.

6. The bioreactor of claim 1, wherein the electromagnetic radiation has one or more wavelengths between about 800 nanometers and about 815 nanometers.

7. The bioreactor of claim 1, wherein the electromagnetic radiation has one or more wavelengths between about 780 nanometers and about 840 nanometers.

8. The bioreactor of claim 1, wherein the power density is at least about 10 mW/cm².

9. The bioreactor of claim 1, wherein the power density is in a range between about 1 mW/cm² and about 15 mW/cm².

10. The bioreactor of claim 1, wherein the power density is in a range between about 1 mW/cm² and about 100 mW/cm².

11. The bioreactor of claim 1, wherein the source comprises an emitter situated outside the reservoir such that electromagnetic radiation from the emitter propagates through one or more walls of the reservoir.

12. The bioreactor of claim 1, wherein the source comprises an emitter situated inside the reservoir.

13. The bioreactor of claim 1, wherein the bioreactor comprises a conduit through which the cell culture moves and wherein the source comprises an emitter situated to irradiate the cells in the conduit with electromagnetic radiation which propagates through one or more walls of the conduit.

14. The bioreactor of claim 1, wherein the at least a portion of the reservoir is covered with a blanket which emits electromagnetic radiation.

15. The bioreactor of claim 14, wherein the blanket comprises woven optical fibers.

16. The bioreactor of claim 1, wherein the source delivers a series of pulses of electromagnetic radiation.

17. The bioreactor of claim 1, wherein the source irradiates the cell culture over at least two periods separated by a period in which the source does not irradiate the cell culture.

18. The bioreactor of claim 1, wherein the source irradiates the cell culture for a period of about 30 seconds to about 2 hours.

19. The bioreactor of claim 1, wherein the source generates a magnetic field applied to the cells.

20. The bioreactor of claim 1, wherein the source generates radio-frequency (RF) radiation which irradiates the cells.

21. A method for enhancing the production of cells or cell-derived products from a bioreactor containing a cell culture, the method comprising delivering an effective amount of electromagnetic energy to cells in the cell culture, wherein delivering the effective amount of electromagnetic energy includes delivering electromagnetic radiation having a power density of at least about 1 mW/cm² within a wavelength bandwidth of less than or equal to approximately 100 nanometers to the cells in the cell culture.

22. The method of claim 21, wherein the wavelength bandwidth is less than or equal to approximately 80 nanometers.

23. The method of claim 21, wherein the wavelength bandwidth is less than or equal to approximately 10 nanometers.

24. The method of claim 21, wherein the electromagnetic radiation has one or more wavelengths between about 630 nanometers and about 910 nanometers.

25. The method of claim 21, wherein the electromagnetic radiation has one or more wavelengths between about 800 nanometers and about 815 nanometers.

26. The method of claim 21, wherein the electromagnetic radiation has one or more wavelengths between about 780 nanometers and about 840 nanometers.

27. The method of claim 21, wherein the power density is at least about 10 mW/cm².

28. The method of claim 21, wherein the power density is in a range between about 1 mW/cm² and about 15 mW/cm².

29. The method of claim 21, wherein the power density is in a range between about 1 mW/cm² and about 100 mW/cm².

30. The method of claim 21, wherein delivering the electromagnetic radiation comprises placing an emitter outside a reservoir holding the cell culture and irradiating the cells with electromagnetic radiation from the emitter, wherein the electromagnetic radiation propagates through one or more walls of the reservoir.

31. The method of claim 21, wherein delivering the electromagnetic radiation comprises placing an emitter inside a reservoir holding the cell culture and irradiating the cells with electromagnetic radiation from the emitter.

32. The method of claim 21, wherein delivering the electromagnetic radiation comprises placing an emitter outside a conduit through which the cell culture moves and irradiating the cells with electromagnetic radiation from the emitter, wherein the electromagnetic radiation propagates through one or more walls of the conduit.

33. The method of claim 21, wherein delivering the electromagnetic radiation comprises covering at least a portion of a reservoir holding the cell culture with a blanket which emits electromagnetic radiation and irradiating the cells with the electromagnetic radiation from the blanket.

34. The method of claim 33, wherein the blanket comprises woven optical fibers.

35. The method of claim 21, wherein delivering the electromagnetic radiation comprises delivering a series of pulses of electromagnetic radiation.

36. The method of claim 21, wherein delivering the electromagnetic radiation comprises at least two periods of irradiation of the cell culture with the electromagnetic radiation separated by a period in which the cell culture is not irradiated by the electromagnetic radiation.

37. The method of claim 21, wherein delivering the electromagnetic radiation comprises irradiating the cell culture for a period of about 30 seconds to about 2 hours.

38. A method for enhancing the production of a vaccine from a bioreactor containing cells in a cell culture, the method comprising delivering an effective amount of electromagnetic energy to cells in the cell culture, wherein delivering the effective amount of electromagnetic energy includes delivering electromagnetic radiation having a power density of at least about 1 mW/cm² within a wavelength bandwidth of less than or equal to approximately 100 nanometers.
